(19) United States
(12) Patent Application Publication
(10) Pub. No.: US 2020/0399006 A1
(43) Pub. Date: Dec. 24, 2020

(54) SYSTEMS AND METHODS FOR TREATING AND PACKAGING CANNABIS AND THE LIKE

(71) Applicants: Mark Cottone, St. Charles, IL (US); Dimitri Rafaeliants, Karmiel (IL); Vadim Vain, Newmarket (CA)

(72) Inventors: Mark Cottone, St. Charles, IL (US); Dimitri Rafaeliants, Karmiel (IL); Vadim Vain, Newmarket (CA)

(21) Appl. No.: 16/908,657

(22) Filed: Jun. 22, 2020

Related U.S. Application Data
(60) Provisional application No. 62/967,181, filed on Jan. 29, 2020.
(60) Provisional application No. 62/864,684, filed on Jun. 21, 2019.

(51) Int. Cl. B65B 55/16; A61L 2/26; A61L 2/10; B65B 1/22; B65B 43/34; B65B 57/02; B65B 51/00; B65B 35/10

(52) U.S. Cl. CPC B65B 55/16; A61L 2/26; A61L 2/10; B65B 1/22; A23L 3/28; B65B 57/02; B65B 51/00; B65B 35/10; A61L 2202/11; B65B 43/34

(57) ABSTRACT

Systems and methods for treating and packaging cannabis and cannabis-like materials, including systems and methods for sanitizing raw cannabis, and systems and methods for packaging cannabis products in receptacles for sale to and/or use by end users of the products. The systems include treatment systems for treating cannabis by utilizing a rotating conveyor and exposing cannabis on the conveyor to ultraviolet radiation. The systems further include packaging systems for filling receptacles with cannabis utilizing a feed funnel that has a tapering interior that slopes toward a funnel exit port, and an auger or a feed tool at the funnel exit port and extending into the receptacle to deliver a desired dose of the cannabis from the feed funnel.

### CROSS-REFERENCE TO RELATED APPLICATIONS

This application claims the benefit of U.S. Provisional Application No. 62/864,684 filed Jun. 21, 2019, and U.S. Provisional Application No. 62/967,181 filed Jan. 29, 2020. The contents of these prior patent documents are incorporated herein by reference.

### FIELD OF THE INVENTION

The present invention generally relates to systems and methods for processing, including treating and packaging, cannabis and cannabis-like materials, and more particularly relates to systems and methods for sanitizing, cleaning, and/or enhancing the potency of raw cannabis using pulsed ultraviolet light, and to systems and methods for packaging cannabis products in receptacles for sale to and/or use by end users of the products.

### BACKGROUND OF THE INVENTION

Many foodstuffs are difficult to sanitize. Such foodstuffs include but are not limited to spices, powders, flours, fruits, grains, vegetables, poultry, and fish. In the handling of many foodstuffs, it is a recognized method of food safety to subject the product to ultraviolet (UV) light. This may be considered a sanitization procedure in many respects. The UV light, when applied properly, is capable of reducing, if not substantially neutralizing, certain pathogens and other deleterious materials and substances that may be on exterior surfaces of the product. In some instances, the UV light may further pass through the surface of the product and into the product, depending on the nature of the product, and whether such interior treatment is desired. This can result in enhancement of aspects of the product.

Pulsed UV light is generally applied from a high intensity light source, a nonlimiting example of which is commercially available from Xenon Corporation of Wilmington, Mass., to generate a pulsed UV light that has been acknowledged by the U.S. Food & Drug Administration as an intervention technology for sanitization.

### SUMMARY OF THE DISCLOSURE

The present invention provides systems and methods for treating and packaging cannabis and cannabis-like materials, including systems and methods for sanitizing, cleaning, and/or enhancing the potency of raw cannabis, and systems and methods for packaging cannabis products in receptacles for sale to and/or use by end users of the products.

According to one aspect of the invention, a treatment system for treating cannabis includes a rotating conveyor having a vertical axis of rotation and an upper surface, means for depositing cannabis on the upper surface of the rotating conveyor, and an ultraviolet light station arranged about the rotating conveyor. The ultraviolet light station produces an exposure field generated by at least one ultraviolet light source that emits ultraviolet radiation, and the rotating conveyor rotates so that cannabis on the upper surface passes through the exposure field for a duration sufficient to have a sanitizing effect on the cannabis exposed to the ultraviolet radiation emitted by the ultraviolet light source.

According to another aspect of the invention, a packaging system for filling receptacles with cannabis includes a hopper for dispensing the cannabis through an outlet thereof, means for comminuting the cannabis within the hopper prior to exiting the outlet, a feed funnel configured to receive the cannabis from the outlet of the hopper, the feed funnel having a tapering interior that slopes toward a funnel exit port, a receptacle for receiving the cannabis from the funnel exit port of the feed funnel, and an auger extending through the funnel exit port and into the receptacle to deliver a desired dose of the cannabis from the feed funnel to the receptacle.

According to yet another aspect of the invention, a packaging system for filling receptacles with cannabis includes a hopper for dispensing the cannabis through an outlet thereof, means for comminuting the cannabis within the hopper prior to exiting the outlet, a feed funnel configured to receive the cannabis from the outlet of the hopper, the feed funnel having a tapering interior that slopes toward a funnel exit port, a receptacle for receiving the cannabis from the funnel exit port of the feed funnel; and a feed tool disposed at the funnel exit port of the feed funnel and extending into the receptacle to deliver a desired dose of the cannabis from the feed funnel to the receptacle.

Other aspects of the invention include methods capable of being performed by systems configured as described above.

These and other aspects, advantages, applications, and features of the invention will be better understood upon consideration of the following detailed description, taken in conjunction with the drawings.

### BRIEF DESCRIPTION OF THE DRAWINGS

FIG. 1 schematically represents a perspective view of a nonlimiting embodiment of a treatment apparatus for generating pulsed UV light capable of treating raw cannabis in accordance with certain aspects of the invention.

FIG. 2 schematically represents a side view of a nonlimiting embodiment of an apparatus for packaging cannabis products in receptacles for sale to and/or use by end users of the products.

FIG. 3 schematically represents a side elevational view of another nonlimiting embodiment of an apparatus for packaging cannabis products in receptacles.

FIG. 4 schematically represents a perspective view of the apparatus of FIG. 1.

FIG. 5 schematically represents an enlarged side elevational view of a filling station of the apparatus of FIG. 3.

FIG. 6 schematically represents a perspective view of the filling station of FIG. 5.

FIGS. 7 through 16 schematically represent perspective views of components of the filling station of FIGS. 5 and 6.

FIG. 17 schematically represents a perspective view of a set-up station for delivering cones to the apparatus of FIG. 3.

### DETAILED DESCRIPTION OF EMBODIMENTS

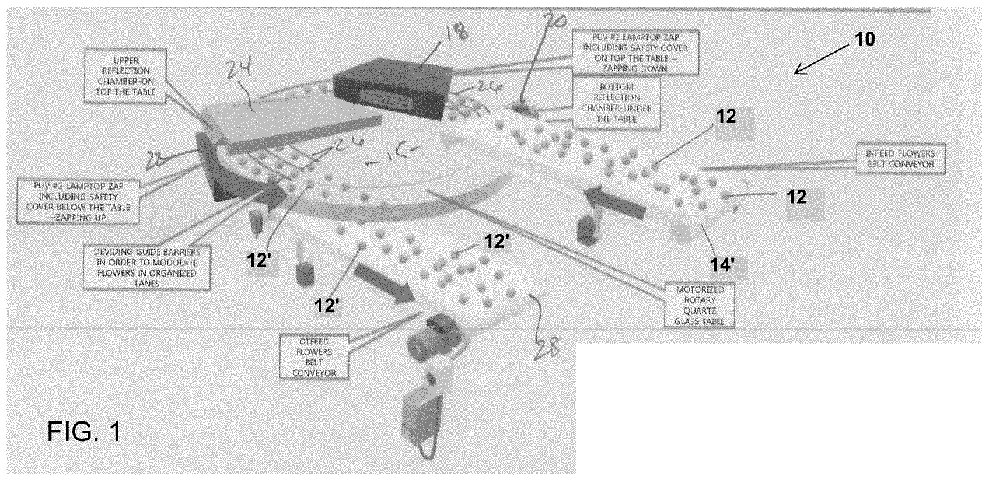

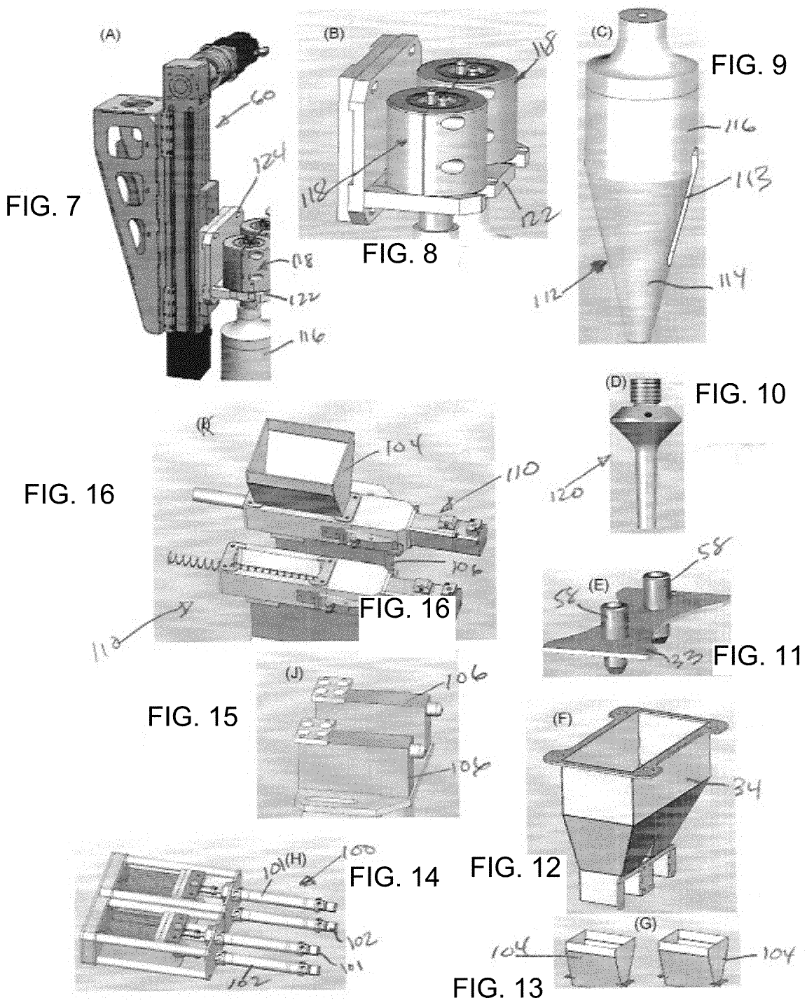

The following disclosure describes various aspects of systems and apparatuses that are schematically represented in FIGS. 1 through 17. One such system is for treating, for example, sanitizing and/or cleaning, raw cannabis (or other loose material) by subjecting the cannabis to pulsed ultraviolet (UV) light. The system may further be operated to increase the potency of cannabis treated therewith. Other aspects include systems and methods for packaging cannabis products in receptacles

In one form, a treatment system is adapted to deliver cannabis (such as [in] seed and flower raw condition) to a surface of a rotating table where the cannabis is subjected to pulsed UV light. The rotating table is preferably compact compared to, for example, a linear conveyor. Cannabis to be treated with pulsed UV light can be initially placed in a supply container, such as a chute or hopper, such that the speed and/or volume of release of the cannabis from a lower end of the container can be controlled. The cannabis is delivered to and spread on the surface of the table, and the table is formed of a transparent material, such as tempered glass or clear quartz, to enable treatment of the cannabis with the pulsed UV light from above the surface as well as below the surface through the table. The speed of rotation of the table can be controlled to provide a suitable time of exposure (dwell time) for the cannabis to the UV light. The table may optionally include vibrational aspects or elements to agitate the cannabis being treated, as well as promote spreading of the cannabis over the surface of the table. The rotation of the table causes the cannabis to be conveyed and passed within an exposure field of one or more pulsed UV light sources that may be arranged about the table to allow for exposure of the cannabis to the UV light. Simultaneous exposure of the cannabis to UV light coming from above and below the table can be achieved by placing the UV light sources above and below the table, or by placing the UV light sources either above or below the table and using reflectors located on the side of the table opposite the light source.

According to another aspect, the pulsed UV light may be in a wavelength range of about 200 to 1100 nm, and most preferably applied in a broadband spectrum across that entire range. Such wavelengths are believed to have a sanitizing effect by destroying molds and yeast spores, rendering them harmless and unable to grow. Further, the broadband UV light is capable of a sanitizing effect by destroying harmful pathogens such as Listeria, Salmonella, Escherichia coli (E-coli), and others. The broadband UV light destroys these spores and pathogens at the DNA level as observed microscopically, rendering the pathogens harmless to humans. Additionally, the higher end of the wavelength range destroys the spores and pathogens at a heat level which creates a much more effective double kill zone, while pulsing of the UV light inhibits any significant change in the ambient temperature of the cannabis being sanitized, thus reducing chances of damaging the product receiving the pulse UV treatment.

Another aspect is a packaging system capable of improving the delivery and packaging of raw dried cannabis products, which may optionally be used in conjunction with the foregoing treatment system and method. Such a packaging system enables cannabis (or other loose material) to be commercially packaged in volume in consumable (e.g., smokable) articles for end users. In one form, the cannabis is packaged in cone receptacles for the cannabis. As described below, the packaging system generally employs, inter alia, a single line or multiple lines that de-nest cone receptacles in a horizontal fashion from a supply, and loads the receptacles through a guide cup, into respective carrier elements (as a nonlimiting example, upwardly-open cylinders on a conveyor chain) at a set-up station. A detection station detects whether a receptacle is in a carrier element to make sure cannabis is not dispensed if a receptacle is not in place. The receptacles, which may be somewhat collapsed, are expanded (re-rounded) with sanitized air at the detection station and preferably later also at a filling station. The cannabis is transported to a filling device comprising a feed funnel, from where it is delivered to the awaiting receptacles in desired weights. The now-filled receptacles then pass to a closing or twisting station, and then progress to an outfeed station as finished product.

The filling device performs pre-weighing through a deduction (subtractive) method, wherein an initial load of cannabis is placed in a filling device hopper and weighed. Cannabis to be dispensed into individual receptacles is then metered out by measuring a reduction in the total weight equal to the desired dose quantity of cannabis to be dispensed to each receptacle.

The filling device may use ultrasonic vibration, and a nano-coating may be applied to an interior surface of the filling device from which the cannabis is funneled to an outlet of the device to provide a “non-stick” surface. A laminar airflow system may also used at the filling head of the device to additionally aid the flow of cannabis through to the receptacles during the filling process. A vacuum system may also be applied to the receptacles during the filling process to enhance compaction and firmness of the cannabis within the receptacles.

Various aspects of the systems described above help to address problems that may arise when processing different varieties of cannabis, some of which are stickier than others. The combination of ultrasonic vibration, nano-coating, and laminar airflow reduces fluctuations during packaging that can occur when attempting to package different varieties and variations of raw cannabis materials.

Turning now to the drawings, FIG. 1 is a perspective and somewhat schematic representation of an embodiment of the treatment system 10 with a rotary table 15, as for treating raw cannabis. Cannabis 12 is represented in FIG. 1 in the form of seeds, leaves and flowers. The invention is not limited to any particular initial condition of the cannabis, but essentially in its raw (unprocessed) condition. Further, while applied in this embodiment for treating to reduce contamination and cleaning, so as to sanitize the product for safe human consumption, the treatment can be used to produce salutary effects in cannabis materials, such as enhanced potency of its psychogenic aspect.

In the nonlimiting embodiment of FIG. 1, the cannabis 12 is loaded onto a supply conveyor 14. The cannabis 12 may be supplied from a supply container (not show), such as a chute or hopper, such that the speed and/or volume of release of the cannabis 12 from the supply container may be appropriately controlled. The manner in which the cannabis 12 is supplied may be of many known types. Cannabis 12 is delivered from the conveyor 14 to the upper surface of a rotary conveyor, referred to herein as a turntable or simply a table 15. The table 15 is generally disk-shaped and made of a transparent material, as nonlimiting examples, tempered glass, clear quartz, or other transparent material. A rigid table 15 is desired, so that it may support its own weight when mounted or suspended from a central axis thereof. However, one can envision supports, such as rollers, upon which the table 15 could rest, thereby extending its radius or providing a thinner table, or both.

In the nonlimiting embodiment of FIG. 1, the table 15 is mounted for rotation by operation of a motor (not shown) connected to a vertical spindle disposed at a central axis of the table 15, defining a vertical axis of rotation of the table 15. The table 15 could also be mounted to be driven from a side roller engaging the circular-shaped edge of the table 15. The motor drive mechanism itself can be of many known types, designed to rotate the table 15 about its vertical axis of rotation. For example, the motor drive can be any constant or variable speed motor, a gas or electric motor (such as a brushed, brushless, or stepper motor, or any other alternate current (AC) or direct current (DC) motor), or any other type of motor. In some embodiments, the motor drive is a 90 or 180 V DC permanent magnet motor. As will be discussed, a controller (not shown) may be used to set the speed (rpm) of the table 15 relative to the amount of dwell time, or exposure, desired for the cannabis 12 (or other material) being treated. The controller may be programmable, or could simply be selection of a desired speed of rotation input to the motor drive. Cannabis 12 dropped from the supply container is also timed to match the rotation speed chosen for the table 15. This timing may be integrated with the controller, or could be set independently of the drive motor.

The cannabis 12 is deposited on the upper surface of the table 15, and as a result of the rotation of the table 15 progresses to ultimately arrive within an exposure field of a first UV light station 18 of potentially multiple UV light stations that may be arranged about the table 15 to allow for exposure of the cannabis 12 to UV light. The UV light station 18 comprises one or more pulsed UV light sources (not shown) mounted within a housing, and preferably at least one reflector to promote emission of the pulsed UV light downward toward the upper surface of the table 15. Preferably, a light reflector unit 20 is located below the table 15 and is aligned with the UV light sources to reflect light that has passed downward through the table 15 upward toward the table 15 and is thus directed at the bottom surfaces of the cannabis 12 on the table 15. Alternatively or in addition, a separate set of UV light sources could be placed below the table 15 to directly expose the bottom of the table 15. The treatment system 10 is represented as further employing a second UV light station 22, with an associated second light reflector unit 24. As depicted herein, the arrangement is reversed from that of the first UV light station 18 and its associated reflector unit 20.

The table 15 may advantageously have dividing guide barriers 26 which are located just above the surface of the table 15, but mounted so as not to move with the table 15. These barriers 26 serve to organize the cannabis 12 into divided lanes. Treated cannabis 12′ then progresses off of the table 15 onto an outfeed conveyor 28, for collection therefrom, as for packaging. In this embodiment, pulsed UV lights are arranged in suitable number, power (lumens) and at appropriate distances to sanitize or otherwise treat the cannabis 12 to a suitable degree. Without limitation, in this embodiment, pulsed UV light(s) may preferably emit broadband radiation of wavelengths covering the range of about 200 to about 1100 nanometers (nm), and may be operated so that the pulse duration is preferably about 2 milliseconds (ms), as approved by the United States Food and Drug Administration for the use of pulsed UV light in the production, processing, and handling of food (21 C.F.R. § 179.41). The UV lights used in this embodiment can be xenon pulsed UV lights, for example, similar to those used in the curing of industrial adhesives or ink. These pulsed UV lights can emit a high-intensity, low-heat beam periodically, for example, every 10 ms. The exposure pulse will be adjusted based upon the kind of product, the desired level of treatment, and the production rate, to name three significant factors to take into consideration.

Though a pulsed exposure is considered to be particularly desirable, a steady or constant exposure may be appropriate in certain circumstances. The source or type of radiant energy could also be modified to suit other circumstances. In general, the concept is to provide a rotary surface upon which cannabis 12 is deposited, the cannabis 12 then traversing a source of energy (light, other electromagnetic energy, etc.) in a desired exposure rate, and then having the treated cannabis 12 discharged from the surface of the rotary table 15, all as the table 15 rotates about its vertical axis of rotation.

The speed of the table 15 and operation of the pulsed UV light(s) in one embodiment are appropriately controlled by a computer processor system. The system 10 may use programmable logic control (PLC) to control the electromechanical attributes of the system 10, such as the motor. For example, the speed of the table 15 and operation of the pulsed UV light(s) can be determined by programmed software stored in a memory and executable by a processor to selectively perform a desired operation when a user so commands.

An additional benefit of fabricating the table 15 from a transparent material is that using reflectors or reflective surfaces may maximize the benefits of the pulsed UV light. Appropriate shielding may be used to protect workers from the pulsed UV light(s). The system 10 may have a ventilation unit for removing ozone gas that may be a byproduct of the UV light treatment. The table 15 may additionally be provided with an apparatus to agitate the product, so as to promote the exposure of all sides and surfaces of the product to the pulsed UV light(s). Such an agitation apparatus may be a shaker, blower, or the like, so as to move the cannabis 12 as it traverses with the table 15.

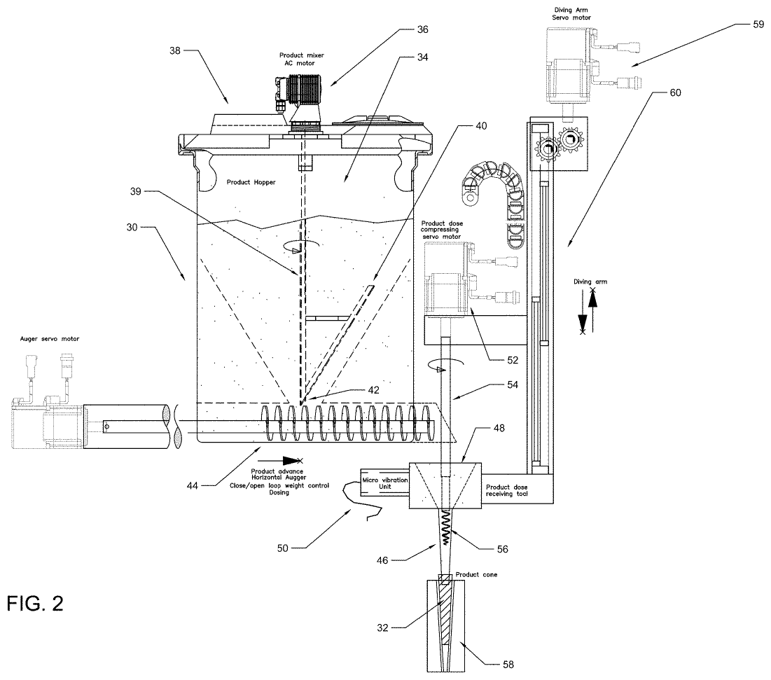

Turning now to FIG. 2, there is depicted a nonlimiting embodiment of a packaging system 30 adapted for filling treated cannabis, now in ground up form, (e.g., chopped or comminuted) into receptacles, represented and hereinafter referred to as cones 32. The cones 32 can be of a type known in the art and are designed for human consumption of their contents. Cannabis (not shown) to be loaded into the contes 32 is placed in a hopper 34. Located at the top of the hopper 34 is a mixer motor 36, which is of readily ascertainable type. In FIG. 2, the motor 36 is mounted to a lid 38. An elongated drive rod 39 extends from the motor 36 and has a mixing arm 40 shown in FIG. 2 as affixed to the bottom of the rod 39 and extending at an angle therefrom. The rod 39 is rotated by the motor 36 to thereby turn the arm 40, which is generally parallel to sloping interior sidewalls of the hopper 34. The arm 40 is operable as a comminuting means that mixes or stirs the cannabis, using gravity feed to an outlet 42 at the lower end of the hopper 34.

A first feed auger 44 receives cannabis 12 from the hopper outlet 42, and advances the cannabis to a cone feed unit 46, which is a filling device that may be of a known type used to fill containers such as with liquid or other fluent material. Cannabis transferred from the first feed auger 44 falls into a feed funnel 48 of the cone feed unit 46. The unit 46 is represented in FIG. 2 as equipped with a micro vibration element 50 to agitate and thereby compact cannabis within the funnel 48. A product dose compressing servo motor 52 has a rotary rod 54 extending therefrom with a spring auger 56 on the end thereof. The feed funnel 48 receives cannabis from the first fed auger 44, and has a tapering interior that slopes toward a funnel exit port of the funnel 48 through which the spring auger 56 extends into a preformed cone 32 held within a fixture 58. In this manner, a desired dose or quantity of cannabis is fed from the funnel 48 via the spring auger 56 into the cone 32, which may be part of a group or plurality of cones 32 held in fixtures 58 for filling seriatim.

Additionally, a diving arm 60 is provided and controlled by a PLC controlled diving arm servo motor 59. The diving arm 60 moves the entire assembly (comprising the servo motor 52, rod 54, funnel 48, auger 56, and cone feed unit 46) thereby traversing the funnel exit port of the cone feed unit 46 down into the cone 32. As the unit 46 deposits its cannabis, it pulls out as it fills cones 32 with the pre-measured amount of cannabis. The servo motor 59 moves the diving arm 60 and cone feed unit 46 as the cannabis is being injected into the cone 32 at a rate that will create the correct packing of cannabis in the cone 32. This portion will be PLC adjustable for the different varieties of cannabis allowing for the correct packing across multiple varieties of cannabis. An air stream may also be incorporate into the cone feed unit 46 to pre-open the cones 32 before the cone feed unit 46 enters the cone 32.

The treatment and packaging systems 10 and 30 of FIGS. 1 and 2 are thus considered to yield a pulsed light treatment system with a relatively compact arrangement in the form of a rotating light table 15 to, in one application of the invention, treat cannabis so as to sanitize the cannabis and clean it by exposure to pulsed UV light sources.

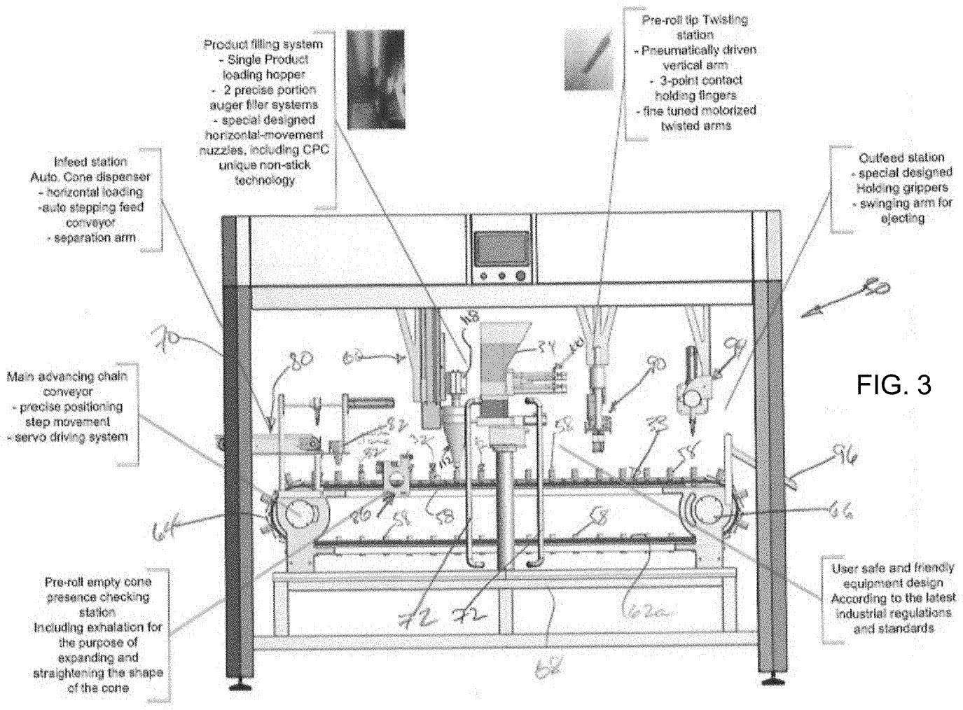

FIGS. 3 through 17 depict another packaging system 30 for receiving and packaging loose material such as cannabis. In one form, the system 30 is used to package cannabis, as in seed and flower raw condition, which may but is not required to have been treated at a pulsed UV light station as described above in reference to FIG. 1. Cannabis is placed in a supply. The supply may preferably be a hopper of a filling device as described above, such that the speed and/or volume of release of the foodstuffs from the bottom of the supply may be appropriately controlled. For convenience, identical reference numerals are used in FIGS. 3 through 17 to denote the same or functionally related elements described for the packaging apparatus 30 of FIG. 2.

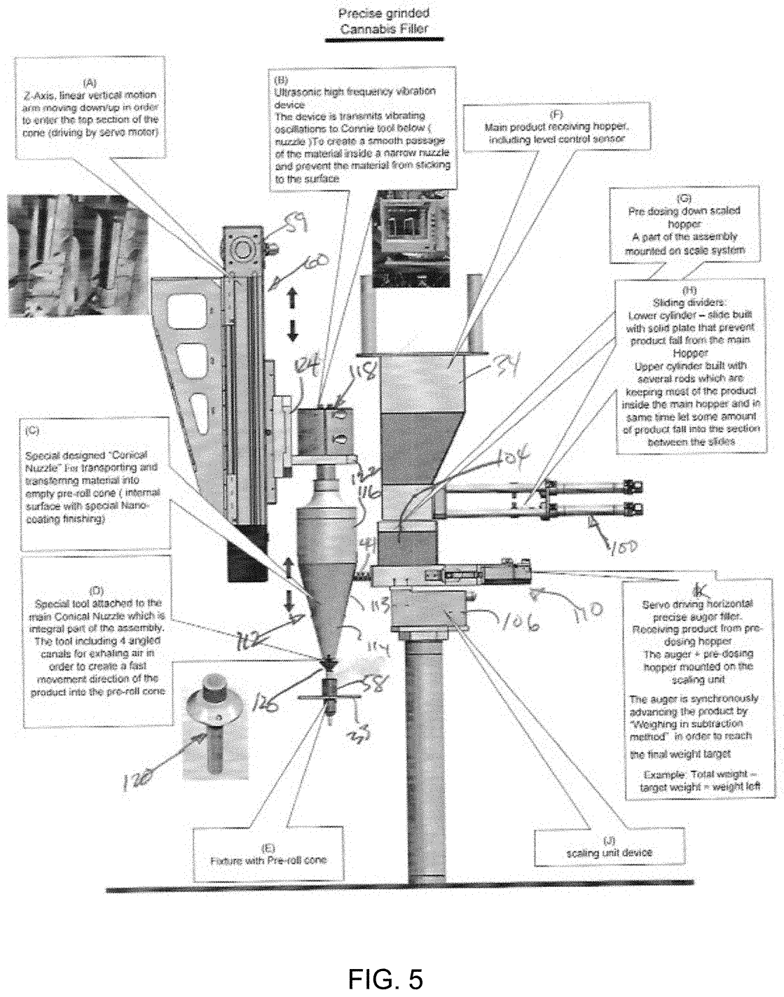

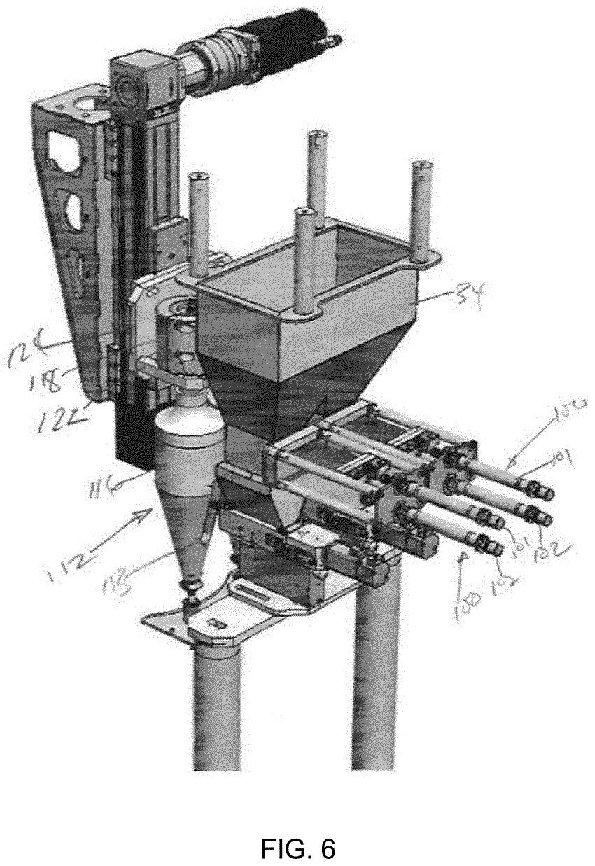

Turning now to FIGS. 5 and 6, the packaging system 30 is adapted for filling cannabis 12 in ground up form, (e.g., chopped or comminuted) into receptacles (cones 32). Though only a few exemplary cones 32 are depicted, these would proliferate along the conveyor 33, which will be discussed in detail hereafter. As previously described for the embodiment of FIG. 2, the cones 32 are known in the art, and are designed for human consumption of its contents, e.g., smoking.

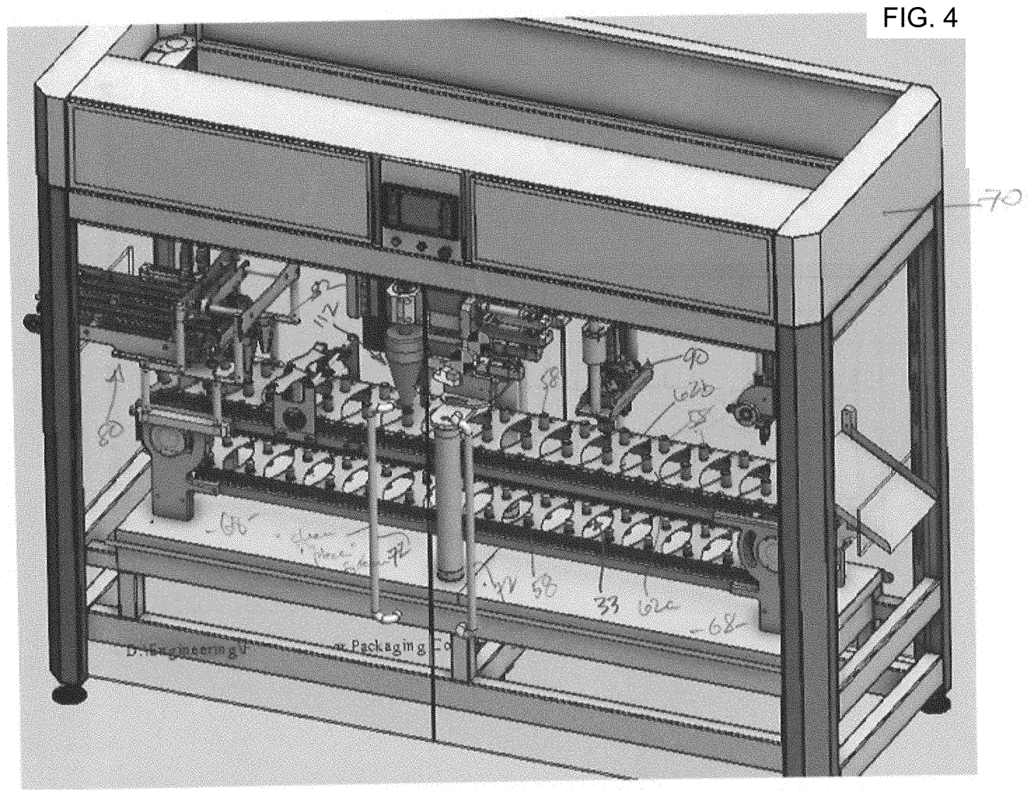

In a brief overview of the main components of a filling station located in the central portion of the packaging system 30 where the cannabis is loaded, cannabis to be loaded is placed in a hopper 34 (see also FIG. 12). Located at the top of the hopper 34 may be a mixer motor such as the motor 36 described in reference to FIG. 2 as mounted to the lid 38 of the hopper 34. Also similar to FIG. 2, an elongated drive rod may extend from the motor and have a mixing arm extending at an angle therefrom so that by turning the arm, the cannabis within the hopper 34 is mixed or stirred, after which the cannabis is gravity fed to an outlet at the lower end of the hopper 34 and is then fed into dividing hoppers 104 (FIG. 13) to feed into individual cone lanes. Two cone lanes or lines are represented on the conveyor 33 shown in FIGS. 3 and 4, so the system 30 is represented as having two dividing hoppers 104 (FIG. 13) fed by the hopper 34.

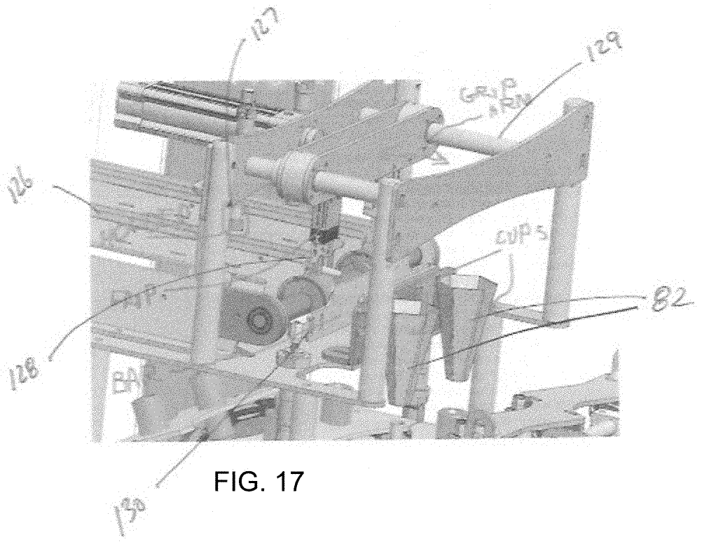

Turning now to FIG. 17 there is depicted a horizontal cone loading station for accurately depositing the cones 32 into fixture 58. A channel device 126 moves a stack of cones 32 against a bar 130, which arrests movement of the cones 32. A holder 127 gently holds the cone 32 that is second in line so a gripper member 128 is able to grab the end of a cone 32 that is first in line to be dispensed. The gripper member 128 moves along a grip arm 129 and deposits the cone 32 into a guide cup (or cone) 82, which then drops into a fixture 58 that has progressed under the guide cup 82.

A first feed auger 44 (FIG. 16) receives cannabis from a dividing hopper 104 (FIG. 12) and advances the cannabis to a small hopper funnel 112 (FIG. 9) having a funnel exit port at which a feed tool 120 (FIG. 10) is disposed, as shown in FIG. 5. The hopper funnel 112 is a filling device generally similar to conventional feed funnels (e.g., 48 of FIG. 2) of known types that have been used to fill containers such as with liquid or other fluent material, but is adapted to better handle cannabis and to cooperate with the tool 120. The hopper funnel 112 and tool 120 collectively define what is referred to herein as a conical nozzle or simply a nozzle. Cannabis transferred from the first feed auger 44 falls into the small hopper funnel 112, which preferably has a ultrasonic vibration device 118 associated therewith to agitate and thereby compact cannabis within the funnel 112.

A desired dose or quantity of cannabis is fed from the funnel end of the hopper funnel 112 via the spring auger (e.g., 56 of FIG. 2) through the funnel exit port of the funnel 112 and into the cone 32 to deliver a desired dose of the cannabis from the funnel 112 to the preformed cone 32. The cone 32 is held within a fixture 58, which may be part of a group or plurality of fixtures 58 that present cones 32 for filling seriatim.

Additionally, a diving arm 60, in the form of a z axis linear vertical motion arm, moves up and down to place a delivery end of the hopper funnel 112 into a cone 32. The diving arm 60 may be controlled by PLC-controlled diving arm servo motor 59, which moves the entire nozzle and its associated elements (funnel 112 and tool 120) down into the cone 32. As the nozzle deposits its cannabis, it is withdrawn as it fills the cone 32 with a pre-measured amount of cannabis. The servo motor 59 moves the diving arm 60, funnel 112 and tool 120 as the cannabis is being injected into the cone 32 at a rate that will create the correct packing of cannabis in the cone 32. This aspect is preferably PLC adjustable for different varieties of cannabis, allowing for the correct packing across multiple varieties of cannabis. An air stream may also be applied by the funnel 112 and/or tool 120 of the nozzle to pre-open the cones 32 before the funnel 112 enters the cone 32. The air stream is preferably a laminar flow that can be advantageously applied to the interior of the funnel 112 to enhance the filling speed and increase the density of the cannabis in the cone 32.

Turning now to FIGS. 3 and 4, the packaging system 30 has a continuous conveyor 33, which is formed in two adjacent lines 62a and 62b, though it should be understood that fewer or more than two lines could be employed. The lines 62a and 62b are joined and present adjacent fixtures 58 across the conveyor 33. At either end of the conveyor 33 are conveyor drive elements. In this instance there is at least one drive motor 64 for a conventional chain conveyor arrangement, and either an opposite drum 66 slaved to the drive motor 64, or it could be another motor which is synchronized with the motor 64. The motor(s) 64 provides rotation of the conveyor 33 and are controlled and timed for precise positioning in a stepwise movement, for example, using a servo driving system. The motor 64 and drum 66 are mounted to a base 68, which in turn is part of an overall assembly fixture or frame 70 that surrounds the packaging system 30 and serves to mount various components of the system 30. The main and central portions of the system 30 described above in reference to FIGS. 5 and 6 are shown generally centered in FIGS. 3 and 4. FIGS. 3 and 4 depict the system 30 as having a clean in place system 72, the elements and operation of which are known in the art, to wash/flush out the interiors of connected components, for cleaning and maintenance.

Turning to the operation of the system 30 of FIGS. 3 through 17, with reference to FIGS. 3 and 4 and going left to right as portrayed in FIG. 3, the conveyor 33 turns clockwise and has a lower run of empty fixtures 58 which turn about the motor 64. An infeed station 80 receives prefabricated cones 32 and serves as a cone dispenser. The infeed station 80 is horizontally loaded and comprises an auto stepping feed conveyor along with a separating arm for separating the cones 32. The separated and now aligned cones 32 are fed in two lines to adjacent guide cups 82, into which the cones 32 are dropped. The guide cups 82 orient the cones 32 into a vertical loading position. The cones 32 are discharged from the guide cups 82 in a timed manner into an awaiting adjacent pair of fixtures 58.

The now-loaded fixtures 58 then progress to a pre-roll detection station 86 that is equipped with a sensor for detecting whether a cone 32 is missing from a fixture 58. The detection station 86 also preferably includes an element which injects a stream of pressurized air into the open top of a cone 32 for the purpose of expanding and straightening the shape of the cone 32 for loading; essentially puffing it into shape.

From the detection station 86, the fixtures 58 with cones 32 therein progress to the central portion of the filling station, where raw cannabis is loaded, such as described above with respect to FIGS. 5 and 6. The now filled cones 32 are then conveyed to a pre-roll tip-twisting station 90. This station 90 moves up and down to place a collar about the open top, or neck, of the cones 32. A three-point contact is made with holding fingers, and a rotary motion is imparted to twist the neck of the engaged cone 32 into a closed position, thus sealing this end of the cone 32.

An outfeed station 94 comprises grasping devices that grip the closed and filled cones 32 similar to the cone loading system shown in FIG. 17, lifting the cones 32 out of their fixtures 58 and, in a swinging motion, places the cones 32 on a ramp 96. The ramp 96 slides the cones 32 into a bin or other container, for further handling, e.g., bulk packaging.

Referring again to FIGS. 5 and 6, raw cannabis is placed as a discrete load into the hopper 34 (FIG. 12). The hopper 34 may be equipped with a level control sensor (not shown) for determining the fill line. A pair of sliding divider mechanisms 100 are located toward the lower end of the hopper 34. The sliding divider mechanisms 100 include upper and lower cylinders 101 and 102 that serve to allow cannabis to flow from the hopper 34 through the cylinders 101 and 102 in a metered manner (FIG. 14).

Cannabis passing through the divider mechanisms 100 then progresses to a dividing hopper 104, which serves as a pre-dosing device as part of a weighing system that includes a scaling unit component 106 (FIGS. 12 and 14). As noted above, dosing, or a load, is determined by weight subtraction from the bulk feed in the hopper 34. As the hopper weight decreases to a predetermined degree (i.e., the desired amount for a cone 32), the load is set.

The load for each of the two lines depicted in FIG and 4 are then fed to a servo driving auger filler section 110 that receives cannabis 12 from the dividing hopper 104. The auger filler section 110 transfers the loads to the hopper funnel 112 of the conical nozzle, which has a funnel-shaped lower body 114 that, similar to the feed funnel 48 of FIG. 2, has a tapering interior that slopes toward the funnel exit port of the funnel 112. The hopper funnel 112 has a vertical groove 113 (FIG. 9), which the auger element of the filler section 110 engages. The tapering interior of the lower body 114 of the funnel 112 is coated with a nano-coating material, as a nonlimiting example, a food grade coating commercially available from NOMA Nanotech SRL, Brugherio, Italy, under the name nanoXHAM. The nano-coating provides a very smooth and low-friction finish so that the cannabis does not tend to adhere to the interior surface of the funnel lower body 114. As such, the funnel 112 is able to dispense cannabis having a wide variety of textures and moisture contents.

Further associated with the hopper funnel 112 is an upper cylindrical portion 116 directly above the lower body 114. Attached to the upper portion 116 is the aforementioned ultrasonic vibration device 118 or, more particularly, a pair of ultrasonic vibration devices 118, as there are two nozzles for the two loads being processed in FIGS. 5 and 6. These are ultrasonic high frequency devices 118 which are used to impart a vibration to the interior of the hopper funnel 112 and its contents to inhibit adhesion of the cannabis 12 to the interior of the lower body 114. Such devices are commercial available from Sonic Italia of Italy and Dukane of St Charles, Ill. USA.

At the end of the funnel lower body 114 of the hopper funnel 112 is the feed tool 120, which is formed to have an internal channel with four angled canals for expressing air to better carry the cannabis into the awaiting cone 32. The airflow is preferably laminar and transports a charge of cannabis along its intended path into a cone 32.

All components of the conical nozzle are mounted on a bracket 122 which is affixed to a carrier plate 124. The carrier plate 124 is part of the diving arm 60, which moves the nozzles up and down for delivery of the cannabis to the cones 32.

In combination, the various aspects and features of the system 30 represented in FIGS. 3 through 17 provide a relatively compact arrangement for loading measured amounts of cannabis, which particularly in its raw forms can vary in degree as to frictional engagement and adhesion to a surface while being dispensed into a receptacle.

While the invention has been described in terms of particular embodiments, it should be apparent that alternatives could be adopted by one skilled in the art. For example, the systems 10 and 30 and their components could differ in appearance and construction from the embodiments described herein and shown in the drawings, functions of certain components of the systems 10 and 30 could be performed by components of different construction but capable of a similar (though not necessarily equivalent) function, process parameters (such as wavelengths, frequencies, and durations) could be modified, and various materials could be used in the fabrication of the systems 10 and 30 and their components. As such, it should be understood that the above detailed description is intended to describe the particular embodiments represented in the drawings and certain but not necessarily all features and aspects thereof, and to identify certain but not necessarily all alternatives to the represented embodiments and described features and aspects thereof. As a nonlimiting example, the invention encompasses additional or alternative embodiments in which one or more features or aspects of a particular embodiment could be eliminated or two or more features or aspects of different embodiments could be combined. Accordingly, it should be understood that the invention is not necessarily limited to any embodiment described herein or illustrated in the drawings, and that the purpose of the above detailed description and the phraseology and terminology employed therein is to describe the illustrated embodiments, and not necessarily to serve as limitations to the scope of the invention. Therefore, the scope of the invention is to be limited only by the following claims.

1. A treatment system for treating cannabis, the treatment system comprising:
a rotating conveyor having a vertical axis of rotation and an upper surface;
means for depositing cannabis on the upper surface of the rotating conveyor; and
an ultraviolet light station arranged about the rotating conveyor, the ultraviolet light station producing an exposure field generated by at least one ultraviolet light source that emits ultraviolet radiation, the rotating conveyor rotating so that cannabis on the upper surface passes through the exposure field for a duration sufficient to have a sanitizing effect on the cannabis exposed to the ultraviolet radiation emitted by the ultraviolet light source.

2. The treatment system of claim 1, wherein the ultraviolet radiation emitted by the ultraviolet light source is a pulsed ultraviolet light.

3. The treatment system of claim 1, further comprising guide members above the rotating conveyor and configured to engage the cannabis, guide the cannabis to the rotating conveyor, and divide the cannabis on the rotating conveyor.

4. The treatment system of claim 1, wherein the rotating conveyor is a rotary table.

5. The treatment system of claim 4, wherein the rotary table is transparent to light from the ultraviolet light source, the treatment system further comprising a reflector on a side of the rotary table opposite to the ultraviolet light source so that ultraviolet radiation that passes through the rotary table is reflected back through the rotary table to the cannabis on the upper surface of the rotary table.

6. The treatment system of claim 1, wherein the exposure field is a limited portion of the upper surface of the rotary table.

7. The treatment system of claim 6, wherein the ultraviolet light station is a first ultraviolet light station located above the rotating conveyor, and the treatment system comprises a second ultraviolet light source located below the rotary table.

8. The treatment system of claim 1, wherein the ultraviolet light source is a pulsed ultraviolet light source which emits broadband ultraviolet radiation consisting of wavelengths within the range of 200 to 1100 nanometers.

9. The treatment system of claim 8, wherein the pulsed ultraviolet light source emits the broadband ultraviolet radiation with pulse durations of about 2 milliseconds.

10. A method for treating raw cannabis, the method comprising:
rotating a conveyor about a vertical axis of rotation;
dispensing cannabis on an upper surface of the conveyor; and
subjecting the cannabis to pulsed ultraviolet radiation as the conveyor rotates about the vertical axis of rotation thereof so that cannabis on the upper surface passes through an exposure field for a duration sufficient to have a sanitizing effect on the cannabis exposed to the ultraviolet radiation.

11. A packaging system for filling receptacles with cannabis, the packaging system comprising:
a hopper for dispensing the cannabis through an outlet thereof;
means for comminuting the cannabis within the hopper prior to exiting the outlet;
a feed funnel configured to receive the cannabis from the outlet of the hopper, the feed funnel having a tapering interior that slopes toward a funnel exit port;
a receptacle for receiving the cannabis from the funnel exit port of the feed funnel; and
an auger extending through the funnel exit port and into the receptacle to deliver a desired dose of the cannabis from the feed funnel to the receptacle.

12. The packaging system of claim 11, wherein the tapering interior of the feed funnel is coated with a nano-coating forming a low-friction finish so that the cannabis does not adhere to the tapering interior of the feed funnel.

13. The packaging system of claim 11, further comprising an ultrasonic vibration device associated with the feed funnel to vibrate the feed funnel and agitate the cannabis therein.

14. A packaging system for filling receptacles with cannabis, the packaging system comprising:
a hopper for dispensing the cannabis through an outlet thereof;
means for comminuting the cannabis within the hopper prior to exiting the outlet;
a feed funnel configured to receive the cannabis from the outlet of the hopper, the feed funnel having a tapering interior that slopes toward a funnel exit port;
a receptacle for receiving the cannabis from the funnel exit port of the feed funnel; and
a feed tool disposed at the funnel exit port of the feed funnel and extending into the receptacle to deliver a desired dose of the cannabis from the feed funnel to the receptacle.

15. The packaging system of claim 14, wherein the tapering interior of the feed funnel is coated with a nano-coating forming a low-friction finish so that the cannabis does not adhere to the tapering interior of the feed funnel.

16. The packaging system of claim 14, further comprising an ultrasonic vibration device associated with the feed funnel to vibrate the feed funnel and agitate the cannabis therein.

17. The packaging system of claim 14, wherein the feed funnel and the feed tool cooperate to deliver laminar air flow to transport the cannabis to the receptacles.

18. The packaging system of claim 14, further comprising:
a loop conveyor having fixtures within which a plurality of the receptacles are received;
a motor drive for turning the loop conveyor about a continuous loop;
an infeed station having a provision of the receptacles for loading into the fixtures, the infeed station including guide cups into which the receptacles are disposed and oriented relative thereto;
a detection station comprising a sensor for determining the presence or absence of receptacles in the fixtures; and
a pressurized air source that injects a stream of pressurized air into the receptacles to expand and straighten the receptacles prior to delivering the dose of the cannabis from the feed funnel to the receptacle.

19. The packaging system of claim 18, wherein the infeed station includes a conveyance for the receptacles which conveys the receptacles horizontally to a position where the receptacles are delivered to a guide cup for vertical admission to one of the fixtures located below the guide cup.

20. The packaging system of claim 14, further comprising a closing station for closing the receptacles after being filled with the cannabis, the closing station being a twisting station which engages and twists an open end of each receptacle into a closed position.
